(19) United States
(12) Patent Application Publication
(10) Pub. No.: US 2026/0203940 A1
(43) Pub. Date: Jul. 16, 2026

(54) IMAGE-TO-3D POSE ESTIMATION VIA DISENTANGLED REPRESENTATIONS

(71) Applicant: Hinge Health, Inc., San Francisco, CA (US)

(72) Inventors: Abel Gonzalez Garcia, Montreal (CA); Maxime Gill-Comeau, Montreal (CA)

(21) Appl. No.: 19/555,652

(22) Filed: Mar. 3, 2026

Related U.S. Application Data
(63) Continuation of application No. PCT/US2024/045191, filed on Sep. 4, 2024.
(60) Provisional application No. 63/580,634, filed on Sep. 5, 2023.

(51) Int. Cl. G06T 7/73; G16H 40/67

(52) U.S. Cl. CPC G06T 7/75; G16H 40/67; G06T 2207/20081

(57) ABSTRACT

Introduced here are computer-implemented platforms (also referred to as “motion monitoring platforms”) that are designed to estimate poses of individuals. A motion monitoring platform can receive an image of an individual in a pose. The motion monitoring platform applies a pose estimation model to the image. The pose estimation model extracts, with a first subnetwork, disentangled representations (e.g., underlying factors hidden in the image data) based on the image. The pose estimation model converts the disentangled representations into two-dimensional (2D) key points and lifts, with a second subnetwork, the 2D key points into a third dimension, resulting in three-dimensional (3D) key points as output. The motion monitoring platform estimates the pose of the individual based on the output. establishing an estimated pose of the individual based on the 3D key points.

### CROSS-REFERENCE TO RELATED APPLICATIONS

This application is a continuation of International Application No. PCT/US2024/045191, titled “IMAGE-TO-3D POSE ESTIMATION VIA DISENTANGLED REPRESENTATIONS” and filed September 4, 2024, which claims priority to US Provisional Application No. 63/580,634, titled “IMAGE-TO-3D POSE ESTIMATION VIA DISENTANGLED REPRESENTATIONS” and filed on September 5, 2023, the entirety of each is incorporated by reference in its entirety.

### TECHNICAL FIELD

Various embodiments concern computer programs and associated computer-implemented techniques for estimating pose of a living body and providing appropriate feedback to promote completion of physical activities.

### BACKGROUND

Pose estimation (also called “pose detection”) is an active area of study in the field of computer vision. Over the last several years, tens – if not hundreds – of different approaches have been proposed in an effort to solve the problem of pose detection. Many of these approaches rely on machine learning due to its programmatic approach to learning what constitutes a pose.

As a field of artificial intelligence, computer vision enables machines to perform image processing tasks with the aim of imitating human vision. Pose estimation is an example of a computer vision task that generally includes detecting, associating, and tracking the movements of a person. This is commonly done by identifying “key points” that are semantically important to understanding pose. Examples of key points include “head,” “left shoulder,” “right shoulder,” “left knee,” and “right knee.” Insights into posture and movement can be drawn from analysis of these key points.

### BRIEF DESCRIPTION OF THE DRAWINGS

FIG. 1 illustrates an example of a network environment that includes a motion monitoring platform.

FIG. 2 illustrates an example of a computing device able to implement a pose estimation model of a motion monitoring platform.

FIGS. 3A-B depict high-level diagrammatic illustrations of models used to determine three-dimensional key points.

FIG. 4 depicts a flow diagram of a process for determining an estimated pose using a pose estimation model.

FIG. 5 includes a block diagram illustrating an example of a processing system in which at least some operations described herein can be implemented.

Various features of the technology described herein will become more apparent to those skilled in the art from a study of the Detailed Description in conjunction with the drawings. Various embodiments are depicted in the drawings for the purpose of illustration. However, those skilled in the art will recognize that alternative embodiments may be employed without departing from the principles of the technology. Accordingly, although specific embodiments are shown in the drawings, the technology is amenable to various modifications.

### DETAILED DESCRIPTION

Over the last several years, significant advances have been made in the field of computer vision. This has resulted in the development of sophisticated pose estimation programs (also called “pose estimators” or “pose predictors”) that are designed to perform pose estimation in either two dimensions or three dimensions. Two-dimensional (“2D”) pose estimators predict the 2D spatial locations of key points, generally through the analysis of the pixels of a single digital image (or simply “image”). Three-dimensional (“3D”) pose estimators predict the 3D spatial arrangement of key points, generally through the analysis of the pixels of multiple images, for example, consecutive frames in a video, or a single image in combination with another type of data generated by, for example, an inertial measurement unit (“IMU”) or Light Detection and Ranging (“LiDAR”) unit.

Pose estimators – both 2D and 3D – continue to be applied to different contexts, and as such, continue to be used to help solve different problems. One problem for which pose estimators have proven to be particularly useful is monitoring the performance of physical activities. Consider, for example, a scenario where an individual is instructed or prompted to perform a physical activity by a computer program. By applying a pose estimator to images of the individual, the computer program can glean insight into performance of the physical activity. Historically, the individual may have instead been asked to summarize her performance of the physical activity (e.g., in terms of difficulty); however, this type of manual feedback tends to be inaccurate and inconsistent. Due to their consistent, programmatic nature, pose estimators allow for more accurate monitoring of performances of physical activities.

This is especially important if the pose estimator is responsible for monitoring physical activities that have meaningful real-world impact, such as on the health and wellness of the individual responsible for performing the physical activities. Exercise therapy is an intervention technique that utilizes physical activities as the principal treatment for addressing the symptoms of musculoskeletal (“MSK”) conditions, such as acute physical ailments and chronic physical ailments. Exercise therapy programs (or simply “programs”) generally involve a plan for performing physical activities during exercise therapy sessions (or simply “sessions”) that occur on a periodic basis. Normally, the purpose of a program is to either restore normal MSK functionality or reduce the pain caused by a physical ailment, which may have been caused by injury or disease.

Programs generally explain, either audibly or visually, how an individual (also called a “user,” “patient,” or “participant”) should perform physical activities to achieve a therapeutic goal. However, individuals can – and often do – struggle to adhere to their respective programs unless consistently engaged. If the individual is not being shown an accurate depiction of how she is positioned (e.g., her pose), she may find it harder to stay engaged with her program or even be unable to participate in the program if the program uses pose to progresses her.

Introduced here is an approach to determining an estimated pose of an individual, which can be used to guide her through performance of physical activities. The approach can leverage a pose estimation model (also called a “pose model,” “pose estimator,” or simply “model”) to output data representing an estimated pose of an individual based on an input image of an individual. As further discussed below, the pose estimation model may perform multiple tasks – namely, determining 2D key points based on an image and then translating those 2D key points into 3D spatial coordinates – that have traditionally been completed using different models in different stages. Simply put, the approach improves pose estimation by a motion monitoring platform. This change in approach is especially useful in implementations where the motion monitoring platform is tasked with estimating pose of an individual in real time as she performs a physical activity, for example, based on analysis of images that are representative of a video feed.

More particularly, the approach leverages a pose estimation model that can include sub-networks. For example, the pose estimation model may initially receive image data of an individual performing a pose, which is input to the first sub-network. The first sub-network can extract 2D disentangled representations from the image data by identifying and disentangling underlying factors hidden in the image data. Each disentangled representation is a one-dimensional (“1D”) vector that represents an x- or y-coordinate of a feature of the image data. The disentangled representations can be converted into 2D key points that represent 2D locations of the individual’s body parts (e.g., limbs, joints, bones, etc.) of the individual’s pose. The second sub-network can lift the 2D key points into 3D key points, which are indicative of a 3D estimated pose of an individual. By determining disentangled representations with one sub-network and lifting with the other sub-network, the pose estimation model is able to determine 3D key points without explicitly relying on image cues. Thus, together, the two sub-networks can process data from an input image to output data representing the estimated pose of the individual.

Using disentangled representations in this manner to estimate 3D pose is an improvement compared to traditional pose estimators that regress output poses using heatmaps. First, heatmaps are computationally heavier than disentangled representations, so the pose estimation model is more efficient using disentangled representations compared to traditional pose estimators that use heatmaps. Said another way, because the pose estimation model uses disentangled representations, the motion monitoring platform can estimate pose in a more computationally “lightweight” manner. Second, the disentangled representations supply the pose estimation model with additional 3D information to complement 2D (e.g., x- and y-coordinate) information used by traditional pose estimators. This additional 3D information may not only improve accuracy, but can also be used to help retrain the pose estimator model as necessary.

For the purpose of illustration, embodiments may be described with reference to exercises that are performed during sessions as part of a program. However, the motion monitoring platform could be designed to monitor performance of other physical activities, such as sporting activities, cooking activities, art activities, and the like. Accordingly, the approach described herein could be used to provide personalized feedback regarding performance of nearly any physical activity.

Moreover, embodiments may be described in the context of computer-executable instructions for the purpose of illustration. However, aspects of the approach could be implemented via hardware or firmware instead of, or in addition to, software. As an example, the motion monitoring platform may be embodied as a computer program that offers support for completing exercises during sessions as part of a program, determines which physical activities are appropriate for a user given performance during past sessions, and enables communication between the user and one or more coaches. The term “coach” may be used to generally refer to individuals who prompt, encourage, or otherwise facilitate engagement by users with the motion monitoring platform. Coaches are generally not healthcare professionals but could be in some embodiments.

### Terminology

References in the present disclosure to “an embodiment” or “some embodiments” mean that the feature, function, structure, or characteristic being described is included in at least one embodiment. Occurrences of such phrases do not necessarily refer to the same embodiment, nor are they necessarily referring to alternative embodiments that are mutually exclusive of one another.

Unless the context clearly requires otherwise, the terms “comprise,” “comprising,” and “comprised of” are to be construed in an inclusive sense rather than an exclusive or exhaustive sense. That is, in the sense of “including but not limited to.” The term “based on” is also to be construed in an inclusive sense. Thus, unless otherwise noted, the term “based on” is intended to mean “based at least in part on.”

The terms “connected,” “coupled,” and variants thereof are intended to include any connection or coupling between two or more elements, either direct or indirect. The connection or coupling can be physical, logical, or a combination thereof. For example, elements may be electrically or communicatively coupled to one another despite not sharing a physical connection.

The term “module” may refer broadly to software, firmware, hardware, or combinations thereof. Modules are typically functional components that generate one or more outputs based on one or more inputs. A computer program may include or utilize one or more modules. For example, a computer program may utilize multiple modules that are responsible for completing different tasks, or a computer program may utilize a single module that is responsible for completing all tasks.

When used in reference to a list of multiple items, the word “or” is intended to cover all of the following interpretations: any of the items in the list, all of the items in the list, and any combination of items in the list.

### Overview of Motion Monitoring Platform

A motion monitoring platform may be responsible for monitoring the motion of an individual (also called a “user,” “patient,” or “participant”) through analysis of images that contain her and are captured as she completes a physical activity. As an example, the motion monitoring platform may guide the user through exercise therapy sessions (or simply “sessions”) that are performed as part of an motion monitoring program (or simply “program”). As part of the program, the user may be requested to engage with the motion monitoring platform on a periodic basis. The frequency with which the user is requested to engage with the motion monitoring platform may be based on factors such as the anatomical region for which therapy is needed, the MSK condition for which therapy is needed, the difficulty of the program, the age of the user, the amount of progress that has been achieved, and the like.

As the user performs exercises, she may be recorded by a camera of a computing device. Normally, the camera is part of the computing device on which the motion monitoring is executed or accessed. For example, in order to initiate a session, the user may initiate a mobile application that is stored on, and executable by, her mobile phone or tablet computer, and the mobile application may instruct the user to position her mobile phone or tablet computer in such a manner that one of its cameras can record her as exercises are performed. Note that, in some embodiments, the camera is part of another computing device. For example, the camera may be included in a peripheral computing device, such as a web camera (also called a “webcam”), that is connected to the computing device. By examining the images that are output by the camera, the motion monitoring platform can monitor performance the exercises by estimating the pose of the user over time.

As mentioned above, the motion monitoring platform could alternatively estimate pose in contexts that are unrelated to healthcare, for example, to improve technique. As an example, the motion monitoring platform may estimate pose of an individual while she completes a sporting activity (e.g., performs a dance move, performs a yoga move, shoots a basketball, throws a baseball, swings a golf club), a cooking activity, an art activity, etc. Accordingly, while embodiments may be described in the context of a user who completes an exercise during a session as part of a program, the features of those embodiments may be similarly applicable to individuals performing other types of physical activities. Individuals whose performances of physical activities are analyzed may be referred to as “users” of the motion monitoring platform, even if these individuals have little to no opportunity to interact with the motion monitoring platform.

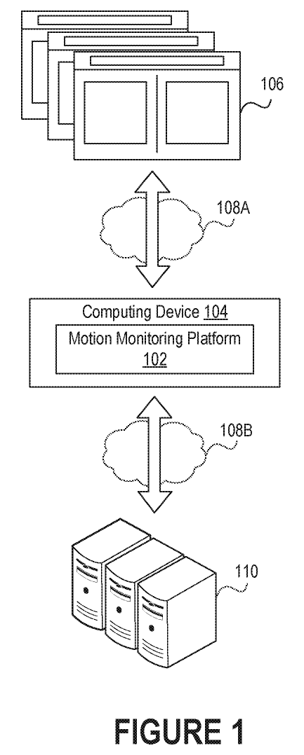

FIG. 1 illustrates a network environment 100 that includes a motion monitoring platform 102 that is executed by a computing device 104. Users can interact with the motion monitoring platform 102 via interfaces 106. For example, users may be able to access interfaces that are designed to guide them through physical activities, indicate progress, present feedback, etc. As another example, users may be able to access interfaces through which information regarding completed physical activities can be reviewed, feedback can be provided, etc. Thus, interfaces 106 may serve as informative spaces, or the interfaces 106 may serve as collaborative spaces through which users and coaches can communicate with one another.

As shown in FIG. 1, the motion monitoring platform 102 may reside in a network environment 100. Thus, the computing device on which the motion monitoring platform 102 is executing may be connected to one or more networks 106A-B. Depending on its nature, the computing device 104 could be connected to a personal area network (“PAN”), local area network (“LAN”), wide area network (“WAN”), metropolitan area network (“MAN”), or cellular network. For example, if the computing device 104 is a mobile phone, then the computing device 104 may be connected to a computer server of a server system 110 via the Internet. As another example, if the computing device 104 is a computer server, then the computing device 104 may be accessible to users via respective computing devices that are connected to the Internet via LANs.

The interfaces 106 may be accessible via a web browser, desktop application, mobile application, or another form of computer program. For example, to interact with the motion monitoring platform 102, a user may initiate a web browser on the computing device 104 and then navigate to a web address associated with the motion monitoring platform 102. As another example, a user may access, via a desktop application or mobile application, interfaces that are generated by the motion monitoring platform 102 through which she can select physical activities to complete, review analyses of her performance of the physical activities, and the like. Accordingly, interfaces generated by the motion monitoring platform 102 may be accessible via various computing devices, including mobile phones, tablet computers, desktop computers, wearable electronic devices (e.g., watches or fitness accessories), virtual reality systems, augmented reality systems, and the like.

Generally, the motion monitoring platform 102 is hosted, at least partially, on the computing device 104 that is responsible for generating the digital images to be analyzed, as further discussed below. For example, the motion monitoring platform 102 may be embodied as a mobile application executing on a mobile phone or tablet computer. In such embodiments, the instructions that, when executed, implement the motion monitoring platform 102 may reside largely or entirely on the mobile phone or tablet computer. Note, however, that the mobile application may be able to access a server system 110 on which other aspects of the motion monitoring platform 102 are hosted.

In some embodiments, aspects of the motion monitoring platform 102 are executed by a cloud computing service operated by, for example, Amazon Web Services®, Google Cloud Platform^{TM}, or Microsoft Azure®. Accordingly, the computing device 104 may be representative of a computer server that is part of a server system 110. Often, the server system 110 is comprised of multiple computer servers. These computer servers can include information regarding different physical activities; computer-implemented models (or simply “models”) that indicate how anatomical regions should move when a given physical activity is performed; computer-implemented templates (or simply “templates”) that indicate how anatomical regions should be positioned when partially or fully engaged in a given physical activity; algorithms for processing image data from which spatial position of anatomical regions can be computed, inferred, or otherwise determined; user data such as name, age, weight, ailment, enrolled program, duration of enrollment, and number of physical activities completed; and other assets.

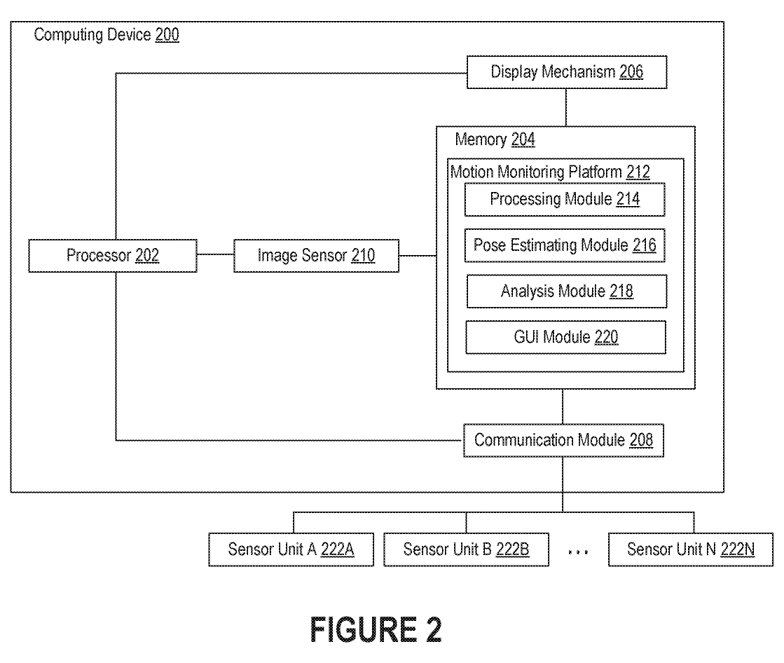

FIG. 2 illustrates an example of a computing device 200 that is able to execute a motion monitoring platform 212. As mentioned above, the motion monitoring platform 212 can facilitate the performance of physical activities by a user, for example, by providing instruction or encouragement based on the user’s estimated pose(s). As shown in FIG. 2, the computing device 200 can include a processor 202, memory 204, display mechanism 206, communication module 208, and image sensor 210A. Each of these components is discussed in greater detail below. The computing device 200 could also include components not shown in FIG. 2, such as an audio output mechanism and audio input mechanism, for example.

Those skilled in the art will recognize that different combinations of these components may be present depending on the nature of the computing device 200. For example, if the computing device 200 is a computer server that is part of a server system (e.g., server system 110 of FIG. 1), then the computing device 200 may not include the display mechanism 206, image sensor 210A, audio output mechanism, or audio input mechanism, though the computing device 200 may be communicatively connectable to another computing device that does include a display mechanism, an image sensor, an audio output mechanism, or an audio input mechanism.

The processor 202 can have generic characteristics similar to general-purpose processors, or the processor 202 may be an application-specific integrated circuit (ASIC) that provides control functions to the computing device 200. As shown in FIG. 2, the processor 202 can be coupled to all components of the computing device 200, either directly or indirectly, for communication purposes.

The memory 204 may be comprised of any suitable type of storage medium, such as static random-access memory (SRAM), dynamic random-access memory (DRAM), electrically erasable programmable read-only memory (EEPROM), flash memory, or registers. In addition to storing instructions that can be executed by the processor 202, the memory 204 can also store data generated by the processor 202 (e.g., when executing the modules of the motion monitoring platform 212) and produced, retrieved, or obtained by the other components of the computing device 200. For example, data received by the communication module 208 from the image sensor 210 (via the processor 202) or sensor units 222A-N may be stored in the memory 204, or data produced by the image sensor 210 may be stored in the memory 204. Note that the memory 204 is merely an abstract representation of a storage environment. The memory 204 could be comprised of actual memory integrated circuits (also referred to as “chips”).

The display mechanism 206 can be any mechanism that is operable to visually convey information to a user. For example, the display mechanism 206 may be a panel that includes light-emitting diodes (“LEDs”), organic LEDs, liquid crystal elements, or electrophoretic elements. In some embodiments, the display mechanism 206 is touch sensitive. Thus, a user may be able to provide input to the motion monitoring platform 212 by interacting with the display mechanism 206. Alternatively, the user may be able to provide input to the motion monitoring platform 212 through some other control mechanism.

The communication module 208 may be responsible for managing communications external to the computing device 200. For example, the communication module 208 may be responsible for managing communications with other computing devices (e.g., server system 110 of FIG. 1, or a camera peripheral such as video camera or webcam). The communication module 208 may be wireless communication circuitry that is designed to establish communication channels with other computing devices. Examples of wireless communication circuitry include 2.4 gigahertz (“GHz”) and 5 GHz chipsets compatible with Institute of Electrical and Electronics Engineers (“IEEE”) 802.11 - also referred to as “Wi-Fi chipsets.” Alternatively, the communication module 208 may be representative of a chipset configured for Bluetooth®, Near Field Communication (“NFC”), and the like. Some computing devices – like mobile phones and tablet computers – are able to wirelessly communicate via separate channels. Accordingly, the communication module 208 may be one of multiple communication modules implemented in the computing device 200. As an example, the communication module 208 may initiate and then maintain one communication channel with a camera peripheral (e.g., via Bluetooth), and the communication module 208 may initiate and then maintain another communication channel with a server system (e.g., via the Internet).

The nature, number, and type of communication channels established by the computing device 200 – and more specifically, the communication module 208 – may depend on the sources from which data is received by the motion monitoring platform 212 and the destinations to which data is transmitted by the motion monitoring platform 212. Assume, for example, that the computing device 200 is representative of a mobile phone or tablet computer that is associated with (e.g., owned by) a user. In some embodiments the communication module 208 may only externally communicate with a computer server, while in other embodiments the communication module 208 may also externally communicate with a source from which to receive image data. The source could be another computing device (e.g., a mobile phone or camera peripheral that includes an image sensor 210B) to which the mobile device is communicatively connected. Image data could be received from the source even if the mobile phone generates its own image data. Thus, image data could be acquired from multiple sources, and these image data may correspond to different perspectives of the user performing a physical activity. Regardless of the number of sources, image data – or analyses of the image data – may be transmitted to the computer server for storage in a digital profile that is associated with the user. The same may be true if the motion monitoring platform 212 only acquires image data generated by the image sensor 210A. The image data may initially be analyzed by the motion monitoring platform 212, and then the image data – or analyses of the image data – may be transmitted to the computer server for storage in the digital profile.

The image sensor 210A may be any electronic sensor that is able to detect and convey information in order to generate images, generally in the form of image data (also called “pixel data”). Examples of image sensors include charge-coupled device (“CCD”) sensors and complementary metal-oxide semiconductor (“CMOS”) sensors. The image sensor 210A may be part of a camera module (or simply “camera”) that is implemented in the computing device 200. In some embodiments, the image sensor 210A is one of multiple image sensors implemented in the computing device 200. For example, the image sensor 210A could be included in a front- or rear-facing camera on a mobile phone. Alternatively, the image sensor 210A may be externally connected to the computing device 200 such that the image sensor 210A captures image data of an environment and sends the image data to the to the motion monitoring platform 212.

For convenience, the motion monitoring platform 212 may be referred to as a computer program that resides within the memory 204. However, the motion monitoring platform 212 could be comprised of software, firmware, or hardware implemented in, or accessible to, the computing device 200. In accordance with embodiments described herein, the motion monitoring platform 212 may include a processing module 214, pose estimating module 216, analysis module 218, and graphical user interface (“GUI”) module 220. These modules can be an integral part of the motion monitoring platform 212. Alternatively, these modules can be logically separate from the motion monitoring platform 212 but operate “alongside” it. Together, these modules may enable the motion monitoring platform 212 to programmatically monitor motion of users during the performance of physical activities, such as exercises, through analysis of images generated by the image sensor 210.

The processing module 214 can process image data obtained from the image sensor 210A over the course of a session. The image data may be used to infer a spatial position or orientation of one or more anatomical regions as further discussed below. The image data may be representation of a series of images. These images may be discretely captured by the image sensor 210A over time, such that each image captured the user at different stages of performing a physical activity. In some embodiments, these images may be representative of frames of a video that is captured by the image sensor 210. In such embodiments, the image data could also be called “video data.”

The image data may be used to infer a spatial position of one or more anatomical regions (e.g., an estimated pose of a user) as further discussed below. For example, the processing module 214 may perform operations (e.g., filtering noise, changing contrast, reducing size) to ensure that the data can be handled by the other modules of the motion monitoring platform 212. As another example, the processing module 214 may temporally align the data with data obtained from another source (e.g., another image sensor) if multiple data are to be used to establish the spatial position of the anatomical regions of interest.

Moreover, the processing module 214 may be responsible for processing information input by users through interfaces generated by the GUI module 220. For example, the GUI module 220 may be configured to generate a series of interfaces that are presented in succession to a user as she completes physical activities as part of a session. On some or all of these interfaces, the user may be prompted to provide input. For example, the user may be requested to indicate (e.g., via a verbal command or tactile command provided via, for example, the display mechanism 206) that she is ready to proceed with the next physical activity, that she completed the last physical activity, that she would like to temporarily pause the session, etc. These inputs can be examined by the processing module 214 before information indicative of these inputs is forwarded to another module.

The pose estimating module 216 (or simply “estimating module”) may be responsible for estimating the pose of the user through analysis of image data, in accordance with the approach further discussed below. Specifically, the estimating module 216 can create, based on an image (e.g., generated by the image sensor 210A or image sensor 210B), a skeletal frame that specifies a spatial position of each of multiple anatomical regions. For example, the estimating module 216 can apply a computer-implemented model (or simply “model”) referred to as a pose estimation model or pose estimator to the image, so as to produce the skeletal frame. In some embodiments the pose estimator is designed and trained to identify a predetermined number of joints (e.g., left and right wrist, left and right elbow, left and right shoulder, left and right hip, left and right knee, left and right ankle, or any combination thereof), while in other embodiments the pose estimator is designed and trained to identify all joints that are visible in the image provided as input. The pose estimator could be a neural network that when applied to the image, analyzes the pixels to independently identify digital features that are representative of each anatomical region of interest.

The analysis module 218 may be responsible for establishing the locations of anatomical regions of interest based on the outputs produced by the estimating module 216. Referring again to the aforementioned examples, the analysis module 218 could establish the locations of joints based on an analysis of the skeletal frame. Moreover, the analysis module 218 may be responsible for determining appropriate feedback for the user based on the outputs produced by the estimating module 216, in accordance with the approach further discussed below. Specifically, the analysis module 218 may determine an appropriate personalized recommendation for the user based on her current position, and a determination as to how her current position compares to a template that is associated with the physical activity that she has been instructed to perform.

Other modules could also be included in some embodiments. For example, the motion monitoring platform 212 may include a training module (not shown) that is responsible for training the pose estimator that is employed by the pose estimating module 216. As another example, the motion monitoring platform 212 may include a template generating module (not shown) that is responsible for generating templates that are used by the analysis module 218 to determine which recommendations, if any, are appropriate for a user given her current position.

Similarly, other components could be implemented in, or accessible to, the computing device 200 in some embodiments. For example, some embodiments of the computing device 200 include an audio output mechanism and/or an audio input mechanism. The audio output mechanism may be any apparatus that is able to convert electrical impulses into sound. One example of an audio output mechanism is a loudspeaker (or simply “speaker”). Meanwhile, the audio input mechanism may be any apparatus that is able to convert sound into electrical impulses. One example of an audio input mechanism is a microphone. Together, the audio output and input mechanisms may enable feedback, such as personalized recommendation as further discussed below, to be audibly provided to the user. Assume, for example, that the user has been instructed to perform a physical activity while being recorded by the image sensor 210A. In such a scenario, the user may be audibly encouraged – in a personalized manner – via the audio output mechanism.

### Overview of Pose Estimation Model

Various attempts have been made to improve pose estimation for use in motion monitoring platforms. Consider, for example, an exercise motion monitoring program that requires exercises be performed by an individual to achieve a therapeutic goal. The individual may perform the exercises, but if the motion monitoring platform inaccurately estimates what poses the individual is doing or the exercises, the individual may not receive useful feedback for the motion monitoring program.

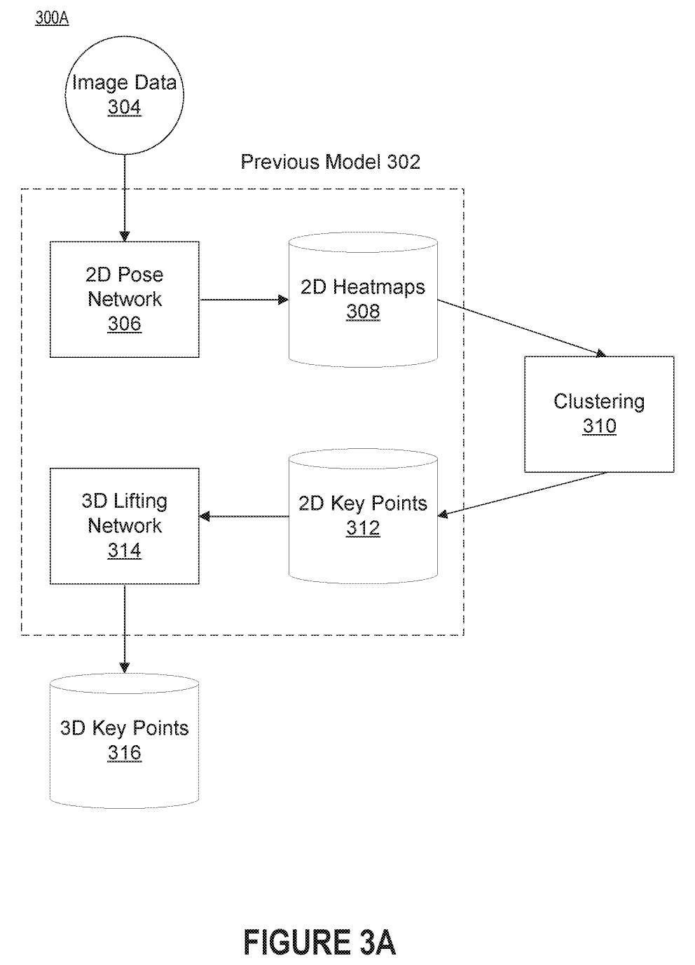

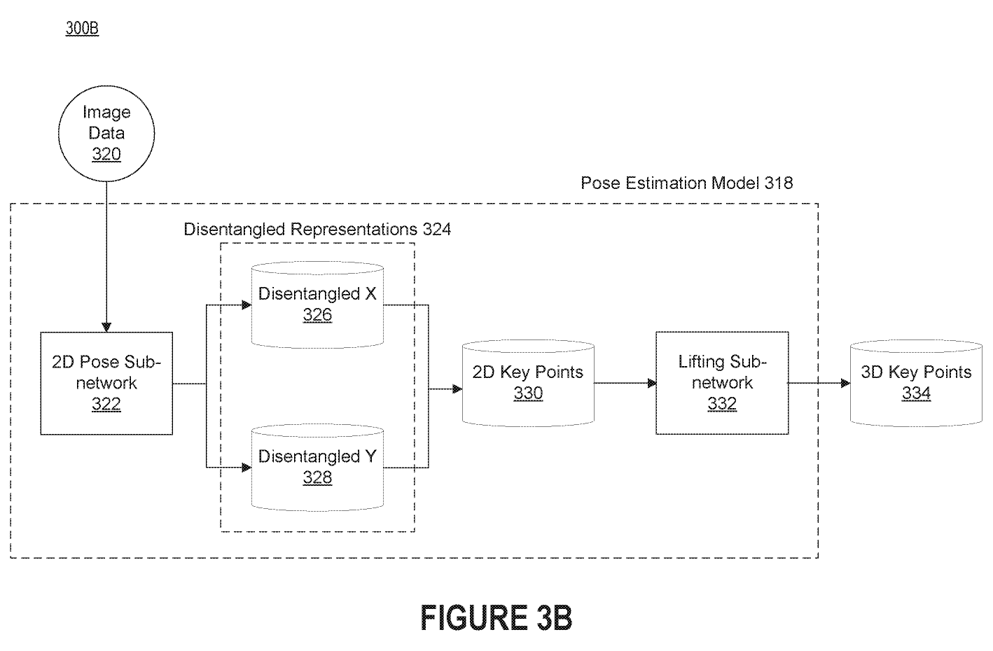

Most conventional 2D pose estimators regress pose using heatmaps to encode all information regarding x- and y-coordinates into a single representation. FIG. 3A depicts a block diagram 300A of a traditional pose estimation model 302 (also called the “previous pose estimation model” or simply “previous model”) that does this using two stages. The previous model 302 receives image data 304 of an individual in an environment as input. For the first stage, the previous model 302 applies a 2D pose network 306 to the image data 304. The 2D pose network outputs 2D heatmaps 308 corresponding to the image data. Each 2D heatmap may correspond to a frame from the image data and include points of 2D data representing the likelihood of a portion of the individual’s pose corresponding to an image pixel located at the same 2D location in the frame.

External to the previous model 302, the 2D heatmaps are clustered 310, which parses the 2D heatmaps into a single representation of the individual’s skeleton (e.g., clustered heatmap data). The clustered heatmap data can be input back to the previous model 302. The previous model 302 uses the clustered heatmap data to determine 2D key points 312 that correspond to the individual in the image data 304, such as 2D points corresponding to the individual’s joints or bones. For the second stage, the previous model 302 inputs the 2D key points 312 to a 3D lifting network 314. At a high level, the 3D lifting network 314 may be representative of a machine learning model that is designed and trained to translate 2D key points into 3D key points. Thus, the 3D lifting network lifts the 2D key points 312 into 3D key points 316. Said another way, the 3D network uses a learned translation scheme to convert the 2D key points from into a 3D coordinate system. The 3D key points can represent the pose of the individual as shown by the image data.

However, conventional approaches (including that described above with respect to FIG. 3A) are computationally inefficient, at least partially due to computational weight of using heatmaps. These approaches also require clustering 310 external to the model, which poses challenge to processing the 2D features by requiring an intermediate stage rather than using the model on its own to convert image data to 3D pose data.

Introduced herein is an approach for estimating poses of an individual using a pose estimation model. The pose estimation model analyzes image data for pose estimation in a two-stage approach that uses disentangled representations. The pose estimation model outputs 3D key points that represent locations of portions of the individual’s body during the exercise. This approach results is advantageous over traditional approaches to pose estimation in that the pose estimation model does not require an external intermediate step to estimate 3D poses and is computationally lighter than the approaches that use heatmaps.

FIG. 3B depicts a block diagram 300B of the pose estimation model 318 that uses disentangled representations 324 to estimate 3D poses of individuals. The pose estimation model 318 includes two sub-networks: a 2D pose sub-network 322 and a lifting sub-network 332. By using two internal sub-networks, the pose estimation model 318 has accuracy similar to a two-stage approach, such as that described in FIG. 33A, but without the need for external clustering 310.

The pose estimation model 318 can receive image data 320 as input. The pose estimation model 318 can apply the 2D pose sub-network 322 to the image data 320. The 2D pose sub-network can be a neural network trained using unsupervised learning to determine disentangled representations based on image data. Disentangled representations 324 are 1D vectors that each correspond to an x- 326 or y-coordinate 328 of the image data 320. The 2D pose sub-network 322 determines each disentangled representation 324 independent to the other disentangled representations 324. Said another way, each disentangled representation is a single latent unit that is sensitive to change in one generative factor while remaining invariant to changes in other factors. For example, a disentangled representation 324 may be sensitive to color of pixels of the image data but not be affected by other features in the image data.

The pose estimation model 318 can apply a differentiable layer to the disentangled representations 324 to determine 2D key points 330 from the disentangled representations. The 2D key points can include an x- and y-coordinate and may include a descriptor describing characteristics of the corresponding image data. The differentiable layer can be a layer within or after the 2D pose sub-network 322 and can use a soft-argmax function (e.g., a normalized exponential function) that converts each disentangled representation 324 (e.g., 1D vector) into a probability of outcomes. At a high level, the differentiable layer may be responsible for implementing one or more arithmetic operations (e.g., to compute the derivative of operations within the 3D pose sub-network 322), and therefore can be used to compute the gradients of the loss function with respect to parameters (i.e., can use backpropagation). For example, the differentiable layer can apply one or more arithmetic operations to the output of the soft-argmax function to determine a location of a maximum value for each disentangled representation 324. The location for each disentangled representation can correspond to an x-coordinate location or a y-coordinate location, which are combined into 2D key points. Said another way, the differentiable layer can convert each disentangled representation into a vector of 2D root-relative (e.g., in a feature-centric coordinate system) key points 330.

The lifting sub-network 332 can receive the 2D key points 330 and converts the 2D key points 330 to 3D key points 334. The lifting sub-network 332 may be a neural network or other machine learning model and can use exemplar-matching, regression, probabilistic inference, or another suitable method to lift the 2D key points 330 into 3D key points 334. The motion monitoring platform 212 receives the 3D key points 334 from the pose estimation model 318 and estimates the individual’s pose based on the 3D key points 334. For example, the 3D key points 334, when mapped together, may result in an outline of the individual’s bones and joints positioned such that her legs are bent at 90-degree angles and her arms are extended forward. In this example, the motion monitoring platform 212 can estimate that the individual is doing a squat.

The 2D pose sub-network 322 and lifting sub-network 332 can be jointly trained for the ultimate task of 3D pose estimation. The motion monitoring platform 212 use disentangled representation learning or another unsupervised learning technique to train the 2D pose sub-network 322. The motion monitoring platform 212 can train the 3D lifting sub-network 332 using supervised, semi-supervised, or unsupervised learning techniques. To train the sub-networks together, the motion monitoring platform 212 can input training data to the 2D pose sub-network and can train the 3D lifting sub-network 332 based on output from the 2D pose sub-network, such that the output of the 2D pose sub-network 322 is input to the 3D lifting network 332 (or differentiable layer in instances where the differentiable layer is not included in the 2D pose sub-network 322) for training. By training the sub-networks jointly, the motion monitoring platform 212 can finely tune every layer of the pose estimation model 318 for higher accuracy and effective alignment compared to conventional techniques for pose estimation.

Using disentangled representations 324 in a pose estimation model 318 to estimate 3D pose in this manner is an improvement compared to traditional pose estimators that regress poses using heatmaps. For one, heatmaps are computationally heavier than disentangled representations and result, so the pose estimation model is more efficient compared to its counterparts that use heatmaps. Further, the disentangled representations supply the pose estimation model with additional 3D information to complement 2D (e.g., x- and y-coordinate) information used by traditional pose estimators, which is a stronger signal for pose estimation model 318 to use to lift the 2D key points 330. Because of these advantages, the pose estimation model 318 is able to predict pose in a single model as accurately as a conventional two-stage model but without the complexity of using an external step (e.g., clustering 310). The pose estimation model is also easier to maintain and deploy given that it is a single model, rather than two models separated by an external step.

### Methodologies for Pose Estimation

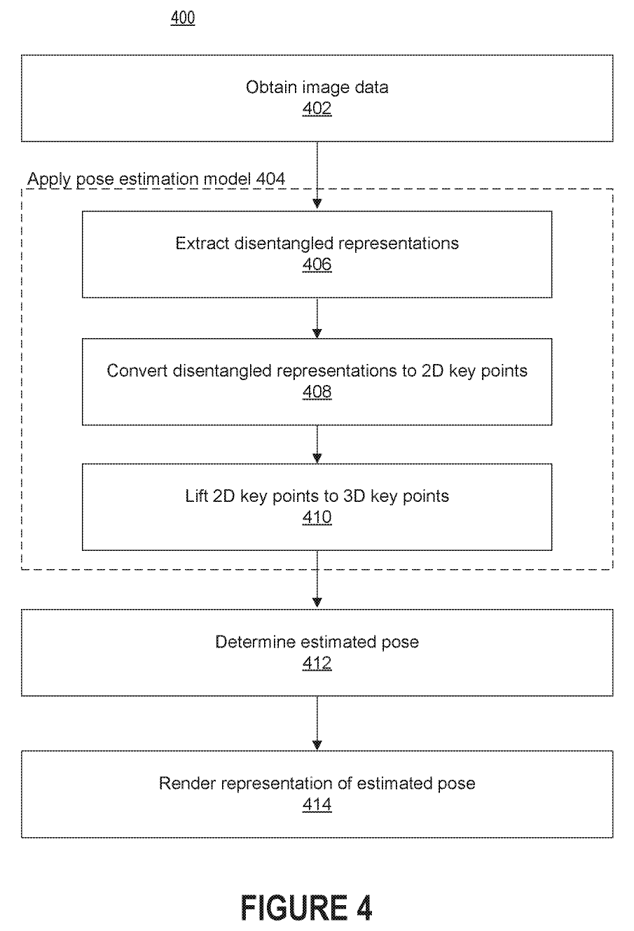

FIG. 4 depicts a flow diagram of a process 400 for determining feedback for a user based on posture indicators, according to one embodiment. The motion monitoring platform 102 can employ the process 400 as part of guiding a user through a set of physical activities for a session. In some embodiments, the motion monitoring platform 102 can perform additional or alternative steps to those shown in FIG. 4 and/or use additional or alternative modules to those described herein to perform the process 400.

Initially, the motion monitoring platform 102 can obtain 402 image data 320 of an individual in an environment. The image data 320 can include images, video, or other visual data. The motion monitoring platform 102 can request image data 320 from an image sensor 210 on a computing device 200 located within the environment of the individual, which causes the image sensor 210 to capture the image data 320 in real-time and send the image data to the motion monitoring platform 102. The motion monitoring platform 102 can also receive image data 320 from a sensor unit 222 external to the computing device 200 running the motion monitoring platform 102. For example, the motion monitoring platform 102 may be running on a couch’s computing device 200 and receive image data 320 from a sensor unit 222 on the individual’s computing device. In some instances, the computing device 200 may receive the image data 320 from an external computing device 200 that received the image data 320 from another external source.

The motion monitoring platform 102 can apply 404 a pose estimation model 318 to the image data 320. The pose estimation model 318 can include two sub-networks that are used to determine 3D key points 334 based on the image data. Key points are distinctive features of the image data that can be detected repeatedly despite changes in scale, resolution, noise, illumination, orientation, and perspective of the image data (e.g., the key points are robust against image transformations and are scale independent). Each key point may be represented by x- and y-coordinates (and a z-coordinate in the case of 3D key points) and may be associated with a descriptor that describes characteristics of the key point. Each descriptor can be a vector of constant length that is a numerical description of the region around the key point and is independent of the position of the key point.

The first sub-network is a 2D pose network 322 can extract 4062D disentangled representations 324 from the image data 320. The first sub-network may be trained using disentangled representation learning, which is a form of unsupervised learning that aims to identify and extract underlying factors of variation in the image data 320 (e.g., the 2D disentangled representations 324). Each 2D disentangled representation is a combination two of 1D vectors that correspond to x- and y-coordinates, respectively, and is a metric that each describes a feature of the image data 320 such that the metric is unaffected by changes to other features in the image data 320. Put another way, a 2D disentangled representation 324 is explainable representation of data that imitates the understanding process of humans when observing a feature (e.g., sematic meaning of features in image data). The 2D disentangled representations 324 can be stored as a first subset corresponding to x-coordinates 326 and a second subset corresponding to y-coordinates 328.

The pose estimation model 318 can convert 408 the disentangled representations 324 into 2D key points 330. The 2D key points 330 can be spatial locations (points) of features (e.g., interesting characteristics) in the image data and may be described in a root-relative coordinate system, meaning that the vectors (e.g., descriptors) of the key point extend from a center of its associated feature’s location. The pose estimation model 318 may convert the disentangled representations using a soft-argmax layer and arithmetic operations. The soft-argmax layer is a normalized exponential function that coverts a vector of K real numbers into a probability distribution of K possible outcomes. The soft-argmax layer may be the final layer of the first sub-network and may normalize the output of the first sub-network before applying arithmetic operations to convert the probability distributions to the 2D key points 330.

The second sub-network is a 3D lifting network 332 that lifts 410 the 2D key points 330 into 3D. The second sub-network can be a neural network with layers that lift input 2D key points 330 into 3D key points 334. The motion monitoring platform 102 may train the neural network using supervised learning, such as by inputting training data to the second sub-network. The training data can be image data labeled with 3D key points 334 and may be entered by an external user to the motion monitoring platform 102. The 3D key points 334 in the labels may have been previously determined by the second sub-network, by another model, or by the user. For example, the motion monitoring platform 102 may display data representing historical 3D key points 334 and corresponding image data 320 to an external user, and the user may interact with a display mechanism 206 connected to or associated with the motion monitoring platform 102 to label the image data 320 with the 3D key points 334. In some instances, the motion monitoring platform 102 can automatically label the image data 320 with the 3D key points 334 before displaying the labeled image data such that an external user can indicate whether the labeling correctly corresponds to features in the image data 320 or not. The motion monitoring platform 102 can retrain the second sub-network periodically, at set times, between uses, etc. using newly labeled output data, which can improve performance of the second sub-network over time.

The motion monitoring platform 102 can receive 3D key points 334 from the second sub-network. The motion monitoring platform 102 uses the 3D key points 334 to determine 412 an estimated pose of the individual depicted in the image data 320. The motion monitoring platform 102 may do so by inputting the 3D key points 334 to a pose engine based on a machine learning model (e.g., a neural network) and/or a mesh creator engine, such as the neural network engine and mesh creator engine described in US Application No. 18/250,845. The motion monitoring platform 102 can store the estimated pose, image data 320, 2D key points 330, 3D key points 334, and/or an identifier of the individual in a local data store, which the motion monitoring platform 102 can access as part of acquiring training data for the second sub-network.

The motion monitoring platform 102 can render interfaces and other visual indicia at a display mechanism 206 of the computing device 200 associated with the individual. For example, the motion monitoring platform 102 can render 414 a representation of the individual performing the estimated pose. The representation may be an avatar of the individual doing a specific yoga pose, knee exercises for physical therapy, a dance move, or any other estimated pose. In another example, the motion monitoring platform 102 can render a visual indicium of the estimated pose. The visual indicium may be a generic avatar performing the estimated pose, an audio or visual description of the estimated pose, a depiction of one or more body parts performing the estimated pose, and the like. The visual indicium may also be a skeletal frame of the individual positioned in the estimated pose.

In some instances, the motion monitoring platform 102 may render the representation or visual indicium to depict the estimated pose along with a series of related poses (e.g., stored in a local database in association with a generic version of the estimated pose or determined by the motion monitoring platform 102 before/after determining the estimated pose), such that the representation or visual indicium depicts how the individual can move to perform the estimated pose. The motion monitoring platform 102 can cause the display mechanism 206 to display the representation and/or visual indicium in a GUI. The motion monitoring platform 102 may also cause the GUI to include other information related to an activity (e.g., physical therapy, tennis, cooking, etc.) associated with the estimated pose.

The process 400 may include additional or alternative steps to those shown in FIG. 4. For example, in some instances, the first and second sub-networks can be trained together. In particular, the first sub-network can be trained using disentangled representation learning, and its output can be input to the second sub-network for training. Each of the sub-networks can receive the output of the second sub-network for unsupervised learning, such that the sub-networks can discover hidden patterns and data groupings to output more accurate key points corresponding to an individual in image data. This allows the sub-networks to learn useful depth information from the image data 320 to inform 3D bone regression with context about joint depth.

### Processing System

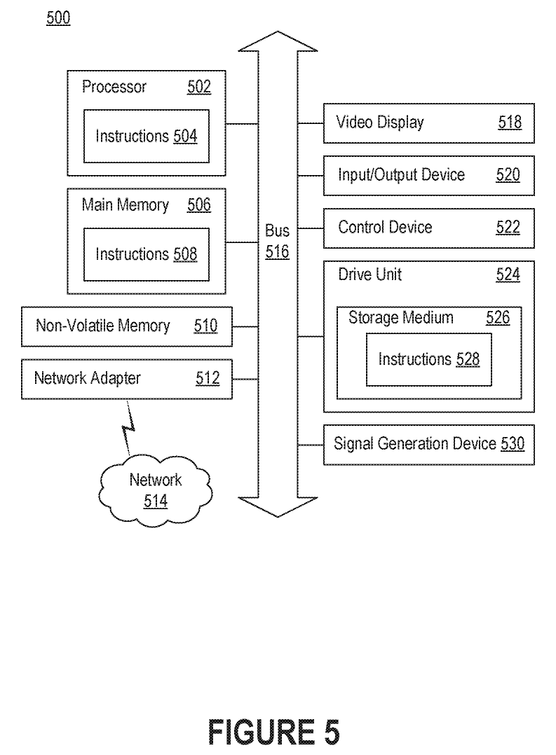

FIG. 5 is a block diagram illustrating an example of a processing system 500 in which at least some operations described herein can be implemented. For example, components of the processing system 500 may be hosted on a computing device that includes a motion monitoring platform (e.g., motion monitoring platform 102 of FIG. 1 or motion monitoring platforms 212 of FIG. 2).

The processing system 500 may include a processor 502, main memory 506, non-volatile memory 510, network adapter 512, video display 518, input/output device 520, control device 522 (e.g., a keyboard or pointing device), drive unit 524 including a storage medium 526, and signal generation device 530 that are communicatively connected to a bus 516. The bus 516 is illustrated as an abstraction that represents one or more physical buses or point-to-point connections that are connected by appropriate bridges, adapters, or controllers. The bus 516, therefore, can include a system bus, a Peripheral Component Interconnect (PCI) bus or PCI-Express bus, a HyperTransport or industry standard architecture (ISA) bus, a small computer system interface (SCSI) bus, a universal serial bus (USB), inter-integrated circuit (I2C) bus, or an Institute of Electrical and Electronics Engineers (IEEE) standard 1394 bus (also referred to as “Firewire”).

While the main memory 506, non-volatile memory 510, and storage medium 526 are shown to be a single medium, the terms “machine-readable medium” and “storage medium” should be taken to include a single medium or multiple media (e.g., a centralized/distributed database and/or associated caches and servers) that store one or more sets of instructions 528. The terms “machine-readable medium” and “storage medium” shall also be taken to include any medium that is capable of storing, encoding, or carrying a set of instructions for execution by the processing system 500.

In general, the routines executed to implement the embodiments of the disclosure may be implemented as part of an operating system or a specific application, component, program, object, module, or sequence of instructions (collectively referred to as “computer programs”). The computer programs typically comprise one or more instructions (e.g., instructions 504, 508, 528) set at various times in various memory and storage devices in a computing device. When read and executed by the processors 502, the instruction(s) cause the processing system 500 to perform operations to execute elements involving the various aspects of the present disclosure.

Further examples of machine- and computer-readable media include recordable-type media, such as volatile memory devices and non-volatile memory devices 510, removable disks, hard disk drives, and optical disks (e.g., Compact Disk Read-Only Memory (CD-ROMS) and Digital Versatile Disks (DVDs)), and transmission-type media, such as digital and analog communication links.

The network adapter 512 enables the processing system 500 to mediate data in a network 514 with an entity that is external to the processing system 500 through any communication protocol supported by the processing system 500 and the external entity. The network adapter 512 can include a network adaptor card, a wireless network interface card, a router, an access point, a wireless router, a switch, a multilayer switch, a protocol converter, a gateway, a bridge, bridge router, a hub, a digital media receiver, a repeater, or any combination thereof.

### Remarks

The foregoing description of various embodiments of the claimed subject matter has been provided for the purposes of illustration and description. It is not intended to be exhaustive or to limit the claimed subject matter to the precise forms disclosed. Many modifications and variations will be apparent to one skilled in the art. Embodiments were chosen and described in order to best describe the principles of the invention and its practical applications, thereby enabling those skilled in the relevant art to understand the claimed subject matter, the various embodiments, and the various modifications that are suited to the particular uses contemplated.

Although the Detailed Description describes certain embodiments and the best mode contemplated, the technology can be practiced in many ways no matter how detailed the Detailed Description appears. Embodiments may vary considerably in their implementation details, while still being encompassed by the specification. Particular terminology used when describing certain features or aspects of various embodiments should not be taken to imply that the terminology is being redefined herein to be restricted to any specific characteristics, features, or aspects of the technology with which that terminology is associated. In general, the terms used in the following claims should not be construed to limit the technology to the specific embodiments disclosed in the specification, unless those terms are explicitly defined herein. Accordingly, the actual scope of the technology encompasses not only the disclosed embodiments, but also all equivalent ways of practicing or implementing the embodiments.

The language used in the specification has been principally selected for readability and instructional purposes. It may not have been selected to delineate or circumscribe the subject matter. It is therefore intended that the scope of the technology be limited not by this Detailed Description, but rather by any claims that issue on an application based hereon. Accordingly, the disclosure of various embodiments is intended to be illustrative, but not limiting, of the scope of the technology as set forth in the following claims.

What is claimed is:

1. A computing device comprising:
a processor;
an image sensor configured to generate an image of an individual in an environment; and
a non-transitory computer-readable storage medium storing instructions that when executed by the processor cause the processor to perform actions comprising:
applying a pose estimation model to an image captured by the image sensor by:
inputting the image to a first sub-network, wherein the first sub-network extracts two-dimensional (2D) disentangled representations based on the image, wherein the 2D disentangled representations are metrics describing features of the environment such that each feature is unaffected by changes of one or more of the other features;
converting the 2D disentangled representations into 2D key points;
inputting the 2D key points to a second sub-network, wherein the second sub-network lifts the 2D key points into a third dimension; and
receiving three-dimensional (3D) key points as output from the second sub-network;
determining, based on the 3D key points, an estimated pose of the individual in the image; and
rendering, via an interface of the computing device, a visual indicum of the individual performing the estimated pose.

2. The computing device of claim 1, wherein each 2D disentangled representation comprises a one-dimensional (1D) vector.

3. The computing device of claim 2, wherein a first subset of the 2D disentangled representations corresponds to x-coordinates and a second subset of the 2D disentangled representations corresponds to y-coordinates.

4. The computing device of claim 1, wherein to convert the 2D disentangled representations to 2D key, the pose estimation model applies a soft-argmax layer to the 2D disentangled representations.

5. The computing device of claim 1, wherein the first sub-network and the second sub-network are jointly trainable.

6. The computing device of claim 1, wherein the first sub-network and the second sub-network are independently trained.

7. The computing device of claim 1, wherein the 2D key points are established in a root-relative coordinate system.

8. A method for estimating a pose of an individual in an image, the method comprising:
applying, to the image, a model that
(i) extracts, with a first subnetwork, two-dimensional (2D) disentangled representations that are indicative of metrics describing features of a surrounding environment such that each feature is unaffected by changes of one or more of the other features,
(ii) converts the 2D disentangled representations into 2D key points that are representative of anatomical landmarks, and
(iii) lifts, with a second subnetwork, the 2D key points into a third dimension, so as to produce three-dimensional (3D) key points as output; and
establishing the pose of the individual based on the 3D key points.

9. The method of claim 8, wherein each 2D disentangled representation comprises a one-dimensional (1D) vector.

10. The method of claim 9, wherein a first subset of the 2D disentangled representations corresponds to x-coordinates and a second subset of the 2D disentangled representations corresponds to y-coordinates.

11. The method of claim 10, wherein to convert the 2D disentangled representations into 2D key points, the model applies a soft-argmax layer to the 2D disentangled representations.

12. The method of claim 10, wherein the first sub-network and the second sub-network are jointly trainable.

13. The method of claim 10, wherein the first sub-network and the second sub-network are independently trained.

14. The method of claim 10, wherein the 2D key points are established in a root-relative coordinate system.

15. A non-transitory computer-readable storage medium storing instructions executable by a processor, wherein the instructions, when executed, cause the processor to perform actions comprising:
receiving, from a digital therapy platform, an image of an individual performing a physical activity in an environment;
applying, to the image, a pose estimation model that
(i) extracts, with a first subnetwork, two-dimensional (2D) disentangled representations that are indicative of metrics describing features of the environment such that each feature is unaffected by changes of one or more of the other features,
(ii) converts the 2D disentangled representations into 2D key points that are representative of anatomical landmarks, and
(iii) lifts, with a second subnetwork, the 2D key points into a third dimension, so as to produce three-dimensional (3D) key points as output;
establishing an estimated pose of the individual based on the three-dimensional (3D) key points; and
causing display, at an interface, a visual indicium of the estimated pose,
wherein the visual indicium represents a skeleton of the individual in the estimated pose.

16. The non-transitory computer-readable storage medium of claim 15, wherein the visual indicium is a skeletal frame of the individual positioned in the estimated pose.

17. The non-transitory computer-readable storage medium of claim 15, wherein each 2D disentangled representation comprises a one-dimensional (1D) vector.

18. The non-transitory computer-readable storage medium of claim 17, wherein a first subset of the 2D disentangled representations corresponds to x-coordinates and a second subset of the 2D disentangled representations corresponds to y-coordinates.

19. The non-transitory computer-readable storage medium of claim 17, wherein to convert the 2D disentangled representations to 2D key points, the pose estimation model applies a soft-argmax layer to the 2D disentangled representations.

20. The non-transitory computer-readable storage medium of claim 17, wherein the 2D key points are established in a root-relative coordinate system.
